(19) United States
(12) Patent Application Publication
(10) Pub. No.: US 2012/0100542 A1
(43) Pub. Date: Apr. 26, 2012

(54) METHOD FOR DETECTION OF TARGET NUCLEIC ACID, AND METHOD FOR TESTING FOR COLON CANCER

(75) Inventors: Tomonori NAGAOKA, Tokyo (JP); Kazue NAKAJIMA, Tokyo (JP); Yasuo TANIGAMI, Kawaguchi-shi (JP)

(73) Assignee: OLYMPUS CORPORATION, Tokyo (JP)

(21) Appl. No.: 13/339,522

(22) Filed: Dec. 29, 2011

Related U.S. Application Data
(63) Continuation of application No. PCT/JP2010/001743, filed on Mar. 11, 2010.

(30) Foreign Application Priority Data
Jul. 6, 2009 (JP) 2009-159848

(51) Int. Cl. C12Q 1/68

(52) U.S. Cl. 435/6.11

(57) ABSTRACT

The present invention provides: a method for easily and simply obtaining highly reliable results of the detection of a target nucleic acid from nucleic acids that are directly recovered from feces; and a method for testing for diseases, particularly colon cancer, by using this method. Specifically, the present invention is a method for detecting an animal-derived target nucleic acid, comprising: (a) a step of collecting a fixed quantity of feces; (b) a step of recovering nucleic acids from the feces that has been collected in the step (a), and preparing a fixed volume of a nucleic acid solution; and (c) a step of dispensing a fixed volume of an aliquot from the nucleic acid solution that has been prepared in the step (b), and detecting the target nucleic acid in the dispensed solution.

### BACKGROUND OF THE INVENTION

1. Field of the Invention

The present invention relates to a method for detecting an animal-derived nucleic acid contained in feces with high precision, and a method for testing for colon cancer by using this method.

Priority is claimed on Japanese Patent Application No. 2009-159848, filed Jul. 6, 2009, the content of which is incorporated herein by reference.

2. Description of the Related Art

With recent progress in gene engineering technologies, gene recombination technologies, and the like, genetic analyses have been expandingly applied to wide ranges of fields such as medical services, academic researches, and industries. For example, diagnoses of diseases such as cancer, infectious diseases attributed to microbes (bacteria), viruses, parasites, and the like, are carried out by collecting RNA or DNA contained in a biological sample such as feces; body fluid including saliva, and blood; mucous membrane including oral mucosa, and uterine mucosa; and mucosal fluid thereof; and then making a comparison of the characteristics of a nucleic acid between samples.

Genetic analysis is usually performed by detecting the presence or absence of a nucleic acid having a nucleotide sequence that is homologous to that of the target gene (a target gene-derived nucleic acid) in a sample, with the target gene being the object of analysis. The target gene-derived nucleic acid in the sample is often amplified for conducting the analysis, in cases where only a very small quantity of specimen is available, like a case of a specimen in a clinical test, or in a case where the nucleic acid concentration in the sample is very low. The most commonly employed method for such nucleic acid amplification is PCR (Polymerase Chain Reaction). For example, in the diagnosis of a genetic disorder, disease susceptibility, cancer, and the like, the method to amplify a target nucleic acid such as an abnormal cell-specific mRNA by PCR for the detection, is often employed.

Colorectal cancer is the top leading cause of death in Japan and the second leading cause of cancer death in the United States. Colorectal cancer is the third leading cause of death in the United States, where about 1,300,000 cases are found and about 50,000 people die from this disease each year. Therefore, measures to deal with cancer must be urgently taken.

In most cases, colorectal cancer starts from a small benign adenoma and is slowly developed into a malignant tumor over several tens of years. Thus, if it is found at an early stage, surgical treatments are so effective that complete recovery would be possible. For example, in a case of a benign adenoma, endoscopic resection which is less invasive than laparotomy is possible. Even in a case of a malignant tumor, if it is in an early stage, endoscopic resection is possible. Furthermore, even in a case of advanced cancer, surgical treatments are often effective. Because of such a slow development process, many chances are left to prevent and intervene this disease. Accordingly, it is possible to reduce the morbidity rate and the mortality rate of colorectal adenoma or tumor by such an early stage detection and resection.

However, currently performed adenoma or cancer detection methods, such as screening test methods for colorectal adenoma or tumor (including a fecal occult blood test, double contrast barium enema, sigmoidoscopy, and total colonoscopy) involve various problems.

For example, the fecal occult blood test is a test to detect a bleeding adenoma or tumor indirectly by checking the presence of blood contained in feces. However, many cases of early stage adenoma or tumor may result in false negatives, and thus the sensitivity can not be said to be sufficient. Moreover, cases of bleeding which occurs not from an adenoma or tumor but from an intestinal tract (such as hemorrhoid) often result in false positives, and thus the specificity can not be said to be high.

The barium enema is an X-ray photographic method in which barium and air are injected from the anus after a thorough laxative pretreatment. This test method can clarify the accurate position and size of cancer, the degree of narrowness of the intestine, and the like. Therefore, it is possible to detect a large-shaped advanced cancer; whereas, on the other hand, the shortcoming is that it is difficult to detect a small-shaped early stage cancer or a flattened cancer.

The sigmoidoscopy and the total colonoscopy are videoscopic methods in which the inside of the intestine is observed after a thorough laxative pretreatment. The pretreatment for these test methods requires the administration of two to three liters of laxative. This imposes an unpleasant burden on the examinee. Furthermore, tearing or perforation of the intestine or other organ might happen during the test. For this reason, these methods are regarded as not appropriate for the screening test.

Because of such concerns, the current test methods as enumerated above can not be said to fulfill necessary and sufficient performance for checking an adenoma or cancer. Therefore, there is a demand for a low invasive test method which can offer high sensitivity and high specificity.

Recently, methods for detecting colon cancer through amplification and analysis of a cancer gene in feces have been disclosed. For example, Patent Document 1 and Non-Patent Document 1 disclose methods for testing for colon cancer through detection of non-apoptotic DNA which are often found in cancer-cell derived nucleic acids, in particular, methods for testing for colon cancer based on the difference in the fragment length of the Alu repeat region, the alphoid repeat region, the p53, or such a cancer-related gene.

In this way, in order to analyze a cancer-cell derived nucleic acid or such a nucleic acid in feces, it is important to recover high quality nucleic acids from feces. For example, as large amounts of residues after digestion and bacteria are contained in feces, a problem arises in that nucleic acids are quite likely to decompose. There is also a problem in that, because nucleic acids recovered from feces include impurities that have been carried over from the feces, the precision of analysis is impaired. For these reasons, methods for recovering high purity nucleic acids from feces while preventing the decomposition and like problems have been developed with the purpose of obtaining more highly reliable results from nucleic acid analyses.

For example, Patent Document 2 discloses a method comprising: cooling a stool down to a temperature below its gel freezing point so as to stabilize the structure of the stool; isolating cells from the stool in this condition, and analyzing DNA extracted therefrom. In addition, as a method to recover RNA from a fecal sample, Non-patent Document 2 discloses a method in which, after removing proteins and such impurities from a fecal sample, RNA is extracted by using phenol and a chaotropic salt, and the thus extracted RNA is recovered through adsorption onto a silica-containing solid support.

There is also a method in which nucleic acids are directly recovered from feces without isolating and collecting cells from feces. For example, Patent Document 3 discloses a method for the preparation of stool samples to analyze a cancer gene in feces. This is a method comprising homogenizing a stool sample at a solvent volume to stool mass ratio of 5:1 at least, and thereafter recovering DNA together with bacterial DNA. Moreover, Patent Document 4 discloses a method comprising: homogenizing a collected stool in the presence of an RNA nuclease inhibitor to prepare a suspension; extracting RNA directly from the thus prepared suspension; and detecting a transcriptional product of a cancer gene, COX-2 (cyclooxygenase-2) gene.

Furthermore, feces contain bile acids, salts thereof, and such substances having inhibitory actions against a nucleic acid amplification reaction such as PCR (Polymerase Chain Reaction) (for example, refer to Non-patent Document 3). For example, the quantity of feces excreted from an adult is supposed to be about 200 to 400 g/day in average. In feces excreted from a healthy subject, bile acids are reportedly contained at 200 to 650 mg/day. In other words, in conversion per gram of feces, about 0.5 mg to 3.25 mg of bile acids are contained in feces of a healthy subject, and a ten times greater amount of bile acids are contained in feces of a patient. Meanwhile, there is also a report teaching that the inhibitory actions of bile salts against PCR appear to be effective when the concentration reaches approximately 50 μg/mL. Accordingly, when extracting nucleic acids from feces and amplifying them by PCR or such a means, it is desirable for improving the amplification efficiency to prevent the carry over of bile salts and such inhibitory substances acting against nucleic acid amplification reactions.

### REFERENCES

Patent Documents
Patent Document 1: Published Japanese Translation No. 2005-514073 of the PCT International Publication
Patent Document 2: Published Japanese Translation No. Hei 11-511982 of the PCT International Publication
Patent Document 3: Published Japanese Translation No. 2002-539765 of the PCT International Publication
Patent Document 4: Japanese Patent (Granted) Publication No. 4134047

Non-Patent Documents
Non-patent Document 1: Boynton and three others, Clinical Chemistry, 2003, Vol. 49, No. 7, pp. 1058-1065
Non-patent Document 2: Alexander and another, 1998, Digestive Diseases and Sciences, Vol. 43, No. 12, pp. 2652 to 2658
Non-patent Document 3: Wilson I G, Applied and Environmental Microbiology, 1997, Vol. 63, pp. 3741 to 3751

In the method disclosed in Patent Document 2, cells are isolated while cooling down the stool sample. This is because, if this isolation operation is conducted without such cooling, accurate detection results would not be obtained due to the denaturation of the stool sample or such reasons. It is important for the effective prevention of such denaturation of the stool sample, to cool it down right after the stool collection. However, in cases of health checkups or such occasions where a stool is collected at home, it is very difficult and unrealistic to cool down the stool sample right after the collection.

Moreover, as conducted in the method disclosed in Patent Document 2 and the method disclosed in Non-patent Document 2, where the method comprises removing impurities from a stool, isolating cells having a target gene, and recovering nucleic acids therefrom, not only is there a problem in that the process of isolating cells is complicated which increments the cost of the test, but also a problem in that the yield of recovered nucleic acids after the process of isolating cells is low and the loss of yield is large because of this process. For this reason, in cases where nucleic acids are recovered from feces, it is desirable to recover them in a mixed state rather than discriminating human-derived cells from bacteria-derived cells.

In the methods disclosed in Patent Documents 3 and 4, nucleic acids are recovered without the process of isolating cells from feces. However, in cases where nucleic acids are directly recovered from feces, there is a problem in that, although larger amounts of impurities in feces are carried over into nucleic acids after the recovery as compared to the method of recovering them after isolating cells, these methods do not give any consideration at all to the carry over of bile acids, bile salts, and such inhibitory substances which inhibit nucleic acid amplification reactions, thus causing insufficiency in the reliability of the results of nucleic acid analyses.

It is an object of the present invention to provide a method for detecting a target nucleic acid serving as the object of analysis in feces, wherein the method is capable of easily and simply obtaining highly reliable detection results without a need of complicated processes to isolate cells, even if nucleic acids have been directly recovered from feces; and a method for testing for a disease, particularly colon cancer, by using this method.

### SUMMARY OF THE INVENTION

The inventors of the present invention have conducted intensive studies to solve the above-mentioned problems. As a result, they discovered that, in a method where nucleic acids directly recovered from feces are used for a reaction to detect a nucleic acid, it is possible to obtain highly reliable detection results by: recovering nucleic acids from feces so that a nucleic acid solution of a preset quantity per quantity of feces can be prepared, and then using a preset volume of the thus recovered nucleic acid solution for the reaction to detect the nucleic acid; rather than quantifying nucleic acids recovered from feces, and then using a fixed quantity of the nucleic acids for the reaction to detect the nucleic acid. This has led to the completion of the present invention.

That is, the present invention provides the following aspects.

(1) A method for detecting a target nucleic acid sequence derived from an animal which excreted feces, from a fecal sample thereof, comprising:

(a) collecting a fixed quantity of feces;

(b) recovering nucleic acids from the feces that has been collected in (a), and preparing a fixed volume of a nucleic acid solution; and

(c) dispensing a fixed volume of an aliquot from the nucleic acid solution that has been prepared in (b), and detecting the target nucleic acid sequence in the dispensed solution.

(2) The method for detecting a target nucleic acid sequence derived from an animal according to the above-mentioned aspect (1), wherein said (a) is (a′) below, and the quantity of the target nucleic acid sequence detected in said (c) is corrected on the basis of the quantity of the feces collected in said (a′):

(a′) collecting feces and measuring the quantity of the feces.

(3) The method for detecting a target nucleic acid sequence derived from an animal according to the above-mentioned aspect (2), wherein the correction of the quantity of the target nucleic acid sequence is conducted by dividing the quantity of the target nucleic acid sequence detected in said (c) by the quantity of the feces collected in said (a′).
(4) The method for detecting a target nucleic acid sequence derived from an animal according to the above-mentioned aspect (1), wherein said (a) is (a′) below, and said (b) is (b′-1) below:

(a′) collecting feces and measuring the quantity of the feces; and (b′-1) recovering nucleic acids from the feces that has been collected in said (a′) and preparing a nucleic acid solution of a volume proportional to the quantity of the feces collected in said (a′).

(5) The method for detecting a target nucleic acid sequence derived from an animal according to the above-mentioned aspect (4), wherein said (b′-1) is (b′-2) below:

(b′-2) mixing the feces that has been collected in said (a′) or the solid content of the feces, with an extraction solution having a volume proportional to the quantity of the feces collected in said (a′), recovering a nucleic acid extracted in the extraction solution, and preparing a nucleic acid solution of a volume proportional to the quantity of the feces collected in said (a′).

(6) The method for detecting a target nucleic acid sequence derived from an animal according to the above-mentioned aspect (1), wherein said (a) is (a′) below, and said (b) is (b″) below:

(a′) collecting feces and measuring the quantity of the feces:

(b″) mixing the feces that has been collected in said (a′) or the solid content of the feces, with an extraction solution having a volume proportional to the quantity of the feces collected in said (a′), extracting a nucleic acid therein, then dispensing a fixed volume of an aliquot from the extraction solution, recovering nucleic acids in the dispensed solution, and preparing a fixed volume of a nucleic acid solution.

(7) The method for detecting a target nucleic acid sequence derived from an animal according to any one of the above-mentioned aspects (1) to (6), wherein the quantity of feces is determined by at least one measurement values selected from the group consisting of a weight, a volume, a volume of the solid content of the feces, and an absorbance.
(8) The method for detecting a target nucleic acid sequence derived from an animal according to any one of the above-mentioned aspects (1) to (7), wherein the target nucleic acid is RNA.
(9) The method for detecting a target nucleic acid sequence derived from an animal according to any one of the above-mentioned aspects (1) to (8), wherein the target nucleic acid is a nucleic acid derived from a human.
(10) The method for detecting a target nucleic acid sequence derived from an animal according to any one of the above-mentioned aspects (1) to (9), wherein the target nucleic acid is a nucleic acid derived from a marker gene of a digestive organ disease.
(11) The method for detecting a target nucleic acid sequence derived from an animal according to any one of the above-mentioned aspects (1) to (9), wherein the target nucleic acid is a nucleic acid derived from a marker gene of cancer.
(12) The method for detecting a target nucleic acid sequence derived from an animal according to any one of the above-mentioned aspects (1) to (9), wherein the target nucleic acid is a nucleic acid derived from a marker gene of colon cancer.
(13) The method for testing for the presence or absence of affection by a disease comprising:

determining whether or not the animal is affected by the disease from the quantity of the target nucleic acid sequence detected on the basis of a preset threshold, the quantity of the target nucleic acid sequence having been determined by collecting a fixed quantity of feces from an animal, recovering nucleic acids from the feces, and preparing a fixed volume of a nucleic acid solution, and dispensing a fixed volume of an aliquot from the nucleic acid solution, and detecting a target nucleic acid sequence in the dispensed solution,

wherein the target nucleic acid sequence is derived from a marker gene of the disease.

(14) A method of testing for the presence or absence of affection by colon cancer comprising:

determining the patient who provided the feces is affected by colon cancer if the quantity of the target nucleic acid detected by the method for detecting a target nucleic acid derived from an animal according to claim 1, is equal to or greater than a preset threshold, and the patient is not affected by colon cancer if the quantity is smaller than said threshold, the quantity of the target nucleic acid having been determined by collecting a fixed quantity of feces from an animal, recovering nucleic acids from the feces, and preparing a fixed volume of a nucleic acid solution, and dispensing a fixed volume of an aliquot from the nucleic acid solution, and detecting a target nucleic acid sequence in the dispensed solution,

wherein the target nucleic acid sequence is derived from a marker gene of colon cancer.

(15) The method for testing for colon cancer according to the above-mentioned aspect (14), wherein the target nucleic acid is a nucleic acid derived from COX-2 (cyclooxygenase-2) gene.

With the method for detecting an animal-derived target nucleic acid of the present invention, it is possible to obtain more highly reliable detection results than ever before, even if nucleic acids that have been directly recovered from feces with lots of impurities are used for a reaction to detect a nucleic acid. Moreover, since the step for quantifying nucleic acids that have been recovered from feces and the step for adjusting the concentration thereof can be skipped, the labor and the cost for the detection of the target nucleic acid can be saved and the risk of contamination and such troubles can be alleviated.

### BRIEF DESCRIPTION OF THE DRAWINGS

FIG. 1A illustrates one aspect of a fecal sample container capable of collecting a fixed quantity of feces.

FIG. 1B illustrates one aspect of the fecal sample container capable of collecting a fixed quantity of feces.

FIG. 1C illustrates one aspect of the fecal sample container capable of collecting a fixed quantity of feces.

FIG. 1D illustrates one aspect of the fecal sample container capable of collecting a fixed quantity of feces.

FIG. 1E illustrates one aspect of the fecal sample container capable of collecting a fixed quantity of feces.

FIG. 2A is a graph showing the expression level (number of copies) of the COX-2 gene in RNA recovered from respective samples in Example 1.

FIG. 2B is a graph showing the expression level (number of copies) of COX-2 gene in RNA recovered from respective samples in Example 1.

FIG. 3 is a graph showing the correlation between the height of a pellet and the volume of the solid content.

FIG. 4A is a graph showing the expression level (number of copies) of COX-2 gene in RNA recovered from respective samples in Example 2.

FIG. 4B is a graph showing the expression level (number of copies) of COX-2 gene in RNA recovered from respective samples in Example 2.

FIG. 5A is a graph showing the expression level (number of copies) of COX-2 gene in RNA recovered from respective samples in Example 3.

FIG. 5B is a graph showing the expression level (number of copies) of COX-2 gene in RNA recovered from respective samples in Example 3.

FIG. 5C is a graph showing the expression level (number of copies) of COX-2 gene in RNA recovered from respective samples in Example 3.

FIG. 5D is a graph showing the expression level (number of copies) of COX-2 gene in RNA recovered from respective samples in Example 3.

FIG. 5E is a graph showing the expression level (number of copies) of COX-2 gene in RNA recovered from respective samples in Example 3.

### BEST MODE FOR CARRYING OUT THE INVENTION

Generally speaking, reaction systems of a variety of reactions for use in nucleic acid analyses are prepared so that a fixed quantity of nucleic acid can be contained therein. This is because of the belief that it would be impossible to secure a sufficient level of detection sensitivity without the presence of an adequate quantity of nucleic acid in the reaction system. The degree of the quantity of nucleic acid to be added to the reaction system has been empirically determined. In addition, in cases of the analysis of a plurality of relatively similar specimens, it is easier, if a fixed quantity of nucleic acid is prepared for use in each reaction system, to conduct a comparative study of the results between respective specimens.

In prior art methods, when using nucleic acids recovered from feces, similarly to with nucleic acids recovered from other biological samples, a fixed quantity of nucleic acids portioned out from the recovered nucleic acids have been used for the reaction system to analyze a nucleic acid. Specifically speaking, for example, in cases where an expression product (mRNA) of a specific gene is adopted as the target nucleic acid, what has been conducted in order to detect the target nucleic acid in feces is that: firstly, RNA is extracted from feces and quantified; then, the yielded RNA is properly diluted to prepare an RNA solution at a fixed concentration; a reverse transcription reaction is performed with use of this RNA solution; and thereafter a nucleic acid amplification reaction such as PCR is conducted with use of the resultant cDNA as a template.

On the other hand, the method for detecting a target nucleic acid of the present invention does not use a fixed quantity of nucleic acids when nucleic acids recovered from feces are to be subjected to a reaction to detect a nucleic acid such as a reverse transcription reaction or a nucleic acid amplification reaction, but is characterized in using nucleic acids recovered from a preset fixed quantity of feces on the basis of the quantity of feces to be supplied to the recovery as the standard. The reason why it becomes possible to obtain more highly reliable detection results by adopting a quantity of feces to be supplied to the recovery as the standard, rather than adopting a quantity of actually recovered nucleic acids as the standard, is not clear; however, it can be considered to be that the quantity of feces-origin inhibitory substances carried over into the reaction system of a reaction to detect a nucleic acid can be kept within a modest level.

In cases where nucleic acids are recovered from feces, the quantity of feces-origin inhibitory substances carried over into the recovered nucleic acids is dependent on the quantity and the condition of feces supplied to the process to recover nucleic acids, and the recovery process itself. The quantity of feces-origin inhibitory substances rarely correlates with the quantity of actually recovered nucleic acids. Fundamentally, feces are heterogeneous. In other words, diverse and various kinds of components are unevenly present in feces. Therefore, even if feces are collected from the same individual, the quantity of recovered nucleic acids may fluctuate depending on the position where the feces are collected. On the other hand, if the recovery condition is the same, the quantity of inhibitory substances carried over into the nucleic acids that have been recovered from a fixed quantity of feces does not fluctuate so much regardless of whether the quantity of the recovered nucleic acids is large or small, and the range of its abundance is within the range of individual difference.

In other words, when the quantity of nucleic acids recovered from feces is sufficient, only a part of the recovered nucleic acids suffice as the quantity of nucleic acids for use in the following reaction to detect a nucleic acid, and thus the quantity of inhibitory substances carried over into the reaction system can be very small. Conversely, when the quantity of the recovered nucleic acids is small, a large part of the recovered nucleic acids has to be used for the reaction to detect a nucleic acid, and thus an excessive quantity of inhibitory substances would be carried over into the reaction system. As a result, the quantity of inhibitory substances carried over into the reaction system fluctuates even if the quantity of nucleic acids added to the reaction system is the same. Therefore, it is highly possible that the result shows no detection of the target nucleic acid if the quantity of recovered nucleic acids is small, whereas the target nucleic acid would be detectable if the quantity of nucleic acids recovered from feces were sufficient (in short, false negative).

In the method for detecting an animal-derived target nucleic acid of the present invention, the quantity of inhibitory substances carried over into the reaction system can be kept within a modest level by adopting a quantity of nucleic acids recovered from a preset fixed quantity of feces, as the quantity of nucleic acids for use in the reaction to detect a nucleic acid. Furthermore, in cases where a large number of specimens have to be handled, the influence of inhibitory substances carried over into the reaction system can be kept within the level of individual difference, by recovering their nucleic acids from respective feces under the same condition, and then using a quantity of nucleic acids which corresponds to the quantity recovered from a preset fixed quantity of feces for the subsequent reaction to detect a nucleic acid.

In the present invention and the description of this application, the term “inhibitory substance” refers to a substance which inhibitorily acts on a general nucleic acid amplification reaction for use in nucleic acid analyses. The inhibitory substance can be specifically exemplified by a bile acid, a bile salt, or the like. Moreover, in the present invention, the term “nucleic acid amplification reaction” refers to an amplification reaction such as PCR in which a nucleic acid is elongated with the aid of a DNA polymerase.

In addition, feces contain large amounts of bacteria such as enterobacteria, and thus the major part of nucleic acids recovered from feces consists of bacteria-derived nucleic acids. In other words, the quantity (weight or concentration) of nucleic acids recovered from feces does not reflect the presence of nucleic acids derived from the animal which excreted the feces. Thus, the use of such a quantity of nucleic acids as the standard means the use of bacteria-derived nucleic acids as the standard. This brings a converse effect to add noise to the detection results. The method for detecting a target nucleic acid of the present invention does not use a quantity of recovered nucleic acids as the standard, and thereby is capable of reducing such noise in the detection results obtained from the reaction to detect the nucleic acid, as compared to the prior art methods.

In particular, it is preferable to apply the present invention to feces collected from patients with colon cancer or suspected subjects (including examinees for whom it is necessary to determine whether or not they have colon cancer). The reason is that: although it is reported that the total amount of bile acids is not different between colon cancer patients and healthy subjects (Mudd D G, et al., Gut, 1980, 1, pp. 587 to 590), actually conducted comparisons on the efficiencies of nucleic acid amplification reactions and the like (for example, refer to Example 4 and the like that, will be described later) show that such inhibitions are more obvious in colon cancer patients than in healthy subjects, which indicates that adverse effects would be imposed on the sensitivity and the specificity of cancer detection if a quantity of recovered nucleic acids is adopted as the standard, similarly to with a test method which uses a usual tissue.

Specifically speaking, the method for detecting an animal-derived target nucleic acid of the present invention (hereunder, may be referred to as the “detection method of the present invention”) is a method for detecting an animal-derived target nucleic acid from a fecal sample, comprising:

(a) collecting a fixed quantity of feces;

(b) recovering a nucleic acid from the feces that has been collected in the (a) and preparing a fixed volume of a nucleic acid solution; and

(c) dispensing a fixed volume of an aliquot from the nucleic acid solution that has been prepared in the (b), and detecting the target nucleic acid in the dispensed solution.

Hereunder is a description of the respective steps.

First, as the step (a), a preset fixed quantity of feces is collected. The quantity of feces to be collected is not particularly limited. However, for example, when it comes to the weight, the quantity is preferably from 10 mg to 1 g. If the quantity of feces is too large, the task to collect it becomes so bothersome and the size of the fecal sample container becomes so large that the handling property and other such properties may be worsened. Conversely, if the quantity of feces is too small, the number of mammalian cells such as exfoliated large intestine cells contained in the feces is so small that a necessary quantity of nucleic acids cannot be recovered, and thus the precision of the analysis of the target nucleic acid may be worsened. In addition, as mentioned above, because feces are heterogeneous, it is preferable to collect a sample from wide areas of feces at the time of collection, so as to avoid the influence of localization of mammalian cells.

The feces to be supplied to the detection method of the present invention is not particularly limited as long as it is collected from an animal, although preferred is feces derived from a mammal, and more preferably from a human. For example, it is preferable to use feces collected from a human for the purpose of routine medical checkups, diagnosis, or such an occasion, although it is possible to use feces of a domestic animal, a wild animal, or the like. Moreover, it is also possible to use feces that has been preserved for a certain period of time after the collection, although it is preferable to use the feces right after the collection. Furthermore, even though it is preferable to use the collected feces right after excretion, it is still possible to use it after a certain period of time after excretion.

In the step (a), the quantity of feces is not particularly limited so long as the quantity can be determined by a measurement value that can enable comparisons between respective specimens. For example, the measurement value may be the weight, the volume (bulk), or the volume of the solid content of feces. The weight and the volume of feces can be measured by usual methods. In addition, the volume of the solid content can be measured by, for example, subjecting feces to a known solid-liquid separation process, such as centrifugal separation or filtration with filters, then removing the liquid component therefrom, and measuring the volume of the remaining residue (solid content) by a usual method.

Moreover, the quantity of feces does not have to be so physically strict as long as comparisons can be between specimens. For example, it is possible to measure the height of a pellet (precipitated solid content) resulting from centrifugal separation of intact feces or a suspension thereof made by adding an appropriate solvent, and to use the thus obtained value as the measurement value of the volume of the solid content of respective feces. In addition, it is also possible to measure the absorbance of a suspension made by suspending feces in an appropriate solvent, or the supernatant thereof, and to use the thus obtained value as the measurement value of the volume of the solid content of respective feces. This utilizes the fact that the absorbance shows a greater value as the solid content is higher in the solution.

The method for collecting feces is not particularly limited, and any method can be employed as long as a predetermined quantity of feces can be collected in the end. For example, it is possible to use a known fecal sample container having a sampling rod capable of collecting a predetermined volume of feces. By charging an extraction solution in advance inside the fecal sample container for collecting feces, it is possible to promptly conduct the nucleic acid extraction step right after the feces collection. In addition, the thus collected feces may be preserved until the step (b) by keeping it suspended in an appropriate storage solution. For example, by charging an appropriate storage solution in advance inside the fecal sample container, and thereafter putting the collected feces into this fecal sample container, it is possible to preserve the feces within the fecal sample container, and it is also possible to transfer it to the place where the nucleic acid extraction and analysis steps are conducted.

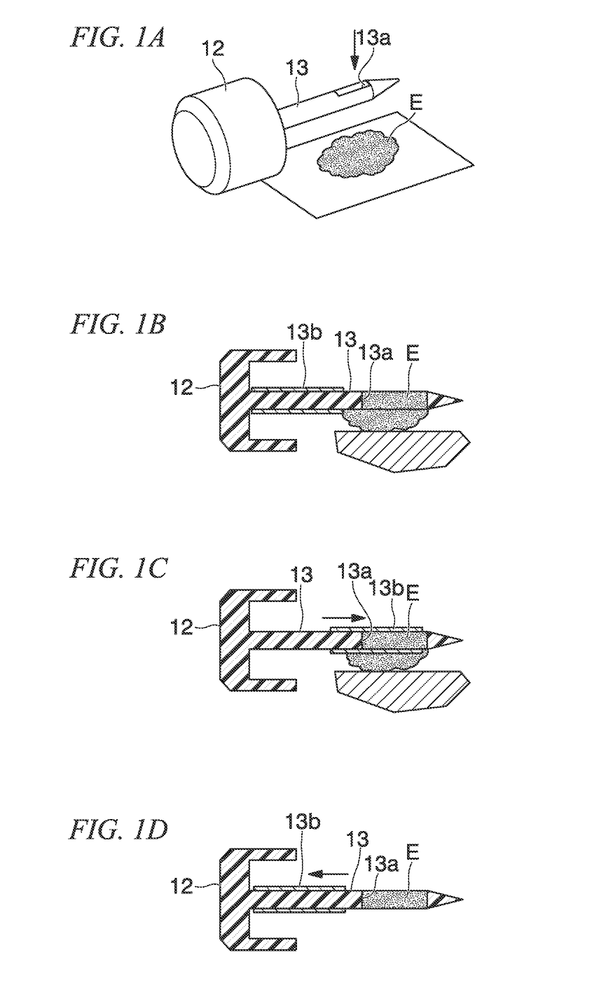

FIG. 1A to FIG. 1E illustrate aspects of a fecal sample container which is integrally unified with a sampling rod capable of collecting a predetermined quantity (volume) of feces. It is a fecal sample container comprising a cap 12 which is integrally unified with a pointed sampling rod 13, and a container mainbody 11. The sampling rod 13 is bored with a hole 13a by which a fixed quantity of feces E can be collected. In addition, attached is a movable lid 13b which can enclose the hole 13a as it slides over the sampling rod 13. As shown in FIG. 1A, firstly, the movable lid 13b is positioned closer to the cap 12 side than to the hole 13a side so that the hole 13a can be completely opened, and thereafter the sampling rod 13 is pressed onto the feces E. By so doing, as shown in FIG. 1B, the hole 13a is filled with the feces E. While keeping this state, the movable lid 13b is slid so that the hole 13a can be enclosed, by which the same volume of feces as that of the inside of the hole 13a can be accurately collected (FIG. 1C). Then, the movable lid 13b is slid back to the original position so that the hole 13a can be completely opened (FIG. 1D). Thereafter, the cap 12 is set in the container mainbody 11 (FIG. 1E). If an appropriate storage solution S has been charged in advance inside the container mainbody 11, it is possible to stably preserve the collected feces until the next step (b) by setting the cap 12, because the feces are immersed in the storage solution S. This kind of fecal sample container can be safely handled at home.

Next, as the step (b), nucleic acids are recovered from the feces that have been collected in the step (a), and a fixed volume of a nucleic acid solution is prepared. In the present invention, the feces is not subjected to any process to separate cells, impurities, and the like, but nucleic acids of all biological species contained in feces, mainly including nucleic acids derived from the animal which excreted the feces and nucleic acids derived from bacteria such as enterobacteria, are extracted and recovered altogether from the feces. Here, examples of such nucleic acids contained in feces can be given by animal-derived nucleic acids, bacteria-derived nucleic acids, and, in addition, nucleic acids derived from foods that have been intaken by the animal and the like.

In the step (b), it suffices if the nucleic acids recovered from feces can be eventually prepared as a nucleic acid solution having a preset fixed quantity. The method for recovering nucleic acids from feces is not particularly limited, and can be appropriately selected and adopted from known methods in the art. The type of nucleic acid to be recovered from feces may be either one or both of DNA and RNA. In the present invention, it is particularly preferable to collect RNA.

For example, nucleic acids can be recovered from the solid content originated from feces (hereunder, may be simply referred to as the “solid content”) by adding an extraction solution to feces or the solid content thereof to effect denaturation of proteins in the feces-origin solid content, then eluting nucleic acids from mammalian cells, enterobacteria, and such cells existing in this solid content, and thereafter recovering the thus eluted nucleic acids.

In cases where a suspension has been prepared by adding a different type of solution, for example, an appropriate storage solution, to the collected feces before the addition of the extraction solution, the solid content is recovered from the suspension and the extraction solution is added to the thus recovered solid content. The recovery of the solid content from the suspension can be conducted by a known solid-liquid separation process, such as centrifugal separation or filtration with filters. It is also possible to add the extraction solution after washing the recovered solid content with an appropriate buffer.

The extraction solution is not particularly limited as long as the solution is capable of denaturing proteins in the solid content, and eluting nucleic acids from mammalian cells, enterobacteria, and such cells existing in this solid content, into the extraction solution. Any type of solution employed in the art can be adopted. For example, a solution prepared by adding, as an active ingredient, a usual compound for use as a protein denaturant, such as a chaotropic salt, an organic solvent, and a surfactant, to an appropriate solvent, can be applied as the extraction solution. It is also possible to combine two or more types of these active ingredients.

The chaotropic salt to serve as an active ingredient of the extraction solution can be exemplified by guanidine hydrochloride, guanidine isothiocyanate, sodium iodide, sodium perchlorate, sodium trichloroacetate, or the like. It is preferable that the surfactant to serve as an active ingredient of the extraction solution is a nonionic surfactant. The nonionic surfactant can be exemplified by Tween 80, CHAPS (3-[3-cholamidopropyl dimethylammonio]-1-propanesulfonate), Triton X-100, Tween 20, or the like. The concentration of the chaotropic salt or the surfactant is not particularly limited as long as nucleic acids can be eluted from the solid content with this concentration. The concentration can be appropriately determined with consideration of the blending ratio of the quantity of feces (the quantity of the solid content) to the extraction solution, the methods for detecting a recovered nucleic acid, and the like.

It is preferable that the organic solvent to serve as an active ingredient of the extraction solution is phenol. The phenol may be either neutral or acidic. When an acidic phenol is used, it is possible to selectively extract RNA into the aqueous layer rather than DNA.

The solvent to be added with such an active ingredient for preparing the extraction solution can be exemplified by phosphate buffer, Tris buffer, or the like. Preferred is an agent in which DNases have been deactivated by high pressure steam sterilization or such a means. Furthermore, more preferred is an agent which contains a proteolytic enzyme such as proteinase K. On the other hand, when it comes to the RNA recovery, for example, a citrate buffer or the like can be used as the extraction solution. However, RNA is a so easily decomposable substance that it is preferable to use a buffer which contains an RNase inhibitor such as guanidine thiocyanate or guanidine hydrochloride.

The quantity of the extraction solution to be added to feces or the solid content thereof is not particularly limited, and can be appropriately determined with consideration of the quantity of feces collected in the step (a), the type of the extraction solution, and the like.

It is preferable to quickly mix the feces or the solid content thereof with the extraction solution. The method of mixing the feces or the like with the extraction solution is not particularly limited as long as the mixing is conducted by a mechanical means. For example, the mixing may be conducted by placing the collected feces or the like in a sealable container where the extraction solution has been charged in advance, sealing the container, and then inverting the container or shaking the container with use of a shaker such as a vortex mixer.

The nucleic acid solution is prepared by recovering nucleic acids that have been eluted from the solid content into the extraction solution, and dissolving the thus recovered nucleic acids with a preset fixed volume of a solvent for preparing the nucleic acid solution. The solvent for use as such a solvent for preparing the nucleic acid solution can be appropriately selected from usual solvents for use in the preparation of a solution that includes purified nucleic acids, with consideration of the following detection method. Such a solvent can be exemplified by purified water or the like.

The recovery of nucleic acids that have been eluted into the extraction solution can be performed by a known means such as ethanol precipitation or cesium chloride ultracentrifugation. The thus recovered nucleic acids are suitably added with water or such an appropriate solvent. By so doing, a fixed volume of a nucleic acid solution can be prepared.

A fixed volume of a nucleic acid solution can also be prepared by adsorbing nucleic acids that have been eluted into the extraction solution, onto an inorganic support, and then, eluting the thus adsorbed nucleic acids from the inorganic support into a fixed volume of a solvent. Regarding the inorganic support to adsorb the nucleic acids, a known inorganic support capable of adsorbing nucleic acids can be adopted. In addition, the shape of the inorganic support is not particularly limited. The shape may be particulate or membranous. Examples of the inorganic support include silica-containing particles (beads) such as silica gel, siliceous oxide, glass, and diatomaceous earth, porous membranes made of nylon, polycarbonate, polyacrylate, and nitrocellulose, and the like. Regarding the solvent to elute the adsorbed nucleic acids from the inorganic support, a usual solvent for use in the elution of nucleic acids from such known inorganic supports can be appropriately adopted with consideration of the type of nucleic acids to be recovered, the following method for analyzing a nucleic acid, and the like. Purified water is particularly preferred as the eluate solvent. Note that it is preferable to wash the inorganic support having nucleic acids adsorbed thereon with an appropriate washing buffer, prior to the elution of the nucleic acids.

Prior to the nucleic acid recovery, the denatured proteins may be removed from the extraction solution in which the nucleic acids are eluted. By removing the previously denatured proteins before the nucleic acid recovery, the quality of the recovered nucleic acids can be improved. The removal of these proteins from the extraction solution can be carried out by a known means. For example, it is possible to remove the denatured proteins by precipitating the denatured proteins through centrifugal separation, and collecting the supernatant alone. In addition, rather than merely conducting the centrifugal separation, it is also possible to remove the denatured proteins even more thoroughly by adding chloroform to the extraction solution, and sufficiently stirring and mixing the mixture using a vortex mixer or the like, before precipitating the denatured proteins through centrifugal separation, and collecting the supernatant alone.

The step (b) can also be carried out by using a commercially available kit such as a nucleic acid extraction kit. This is because commercially available nucleic acid extract kits generally adopt a method for extracting nucleic acids from a predetermined quantity of feces by using a predetermined quantity of an extraction solution, and recovering the nucleic acids in the form of a predetermined quantity of a nucleic acid solution.

Furthermore, as the step (c), a fixed volume of an aliquot is dispensed from the nucleic acid solution that has been prepared in the step (b), and the target nucleic acid in the dispensed solution is detected. In other words, the fixed volume of the dispensed solution is used for the reaction to detect a nucleic acid such as a reverse transcription reaction or a nucleic acid amplification reaction. In this way, by using a preset volume of the nucleic acid solution for the reaction to detect a nucleic acid, irrespective of the concentration of the nucleic acid solution prepared in the step (c), an excessive quantity of feces-origin inhibitory substances can be kept from being carried over into the reaction system of the reaction to detect a nucleic acid. Furthermore, there is no need of the step for measuring the concentration of the nucleic acid solution through UV spectrometry or such a means and thereafter diluting the nucleic acid solution at a fixed concentration, which the prior art methods have required. Hence, the time and labor required for the detection process can be saved, and the risk of contamination and consequent decomposition of nucleic acids can be alleviated.

For example, in health checkups, it is usual that the examinee collects feces by roughly weighing its quantity, which often makes it difficult to collect a preset fixed quantity of feces. Even if the quantity of collected feces varies in such a way, it is possible to obtain the effect of the present invention by correcting the quantity of the finally detected target nucleic acid on the basis of the quantity of the feces. The reason is that: the effect of the present invention can be supposed to be obtained by the effect achieved by equally setting the quantity of feces-origin inhibitory substances which are carried over with the nucleic acids into the reaction to detect a nucleic acid, at a quantity to be carried over from a fixed quantity of feces, because the nucleic acid solution to be supplied to the reaction to detect the nucleic acid is prepared so that all nucleic acids recovered from the fixed quantity of feces can be contained therein.

Specifically speaking, firstly, as the step (a′), an appropriate quantity of feces is collected, and the quantity of the feces is measured. The measurement of the quantity of feces is not particularly limited so long as the quantity can be determined by a measurement value that can enable comparisons between respective specimens, similarly to with the step (a). It is particularly preferable to measure one or more values selected from the group consisting of the weight, the volume (bulk), the volume of the solid content, and the absorbance of feces.

Then, the collected feces is subjected to the steps (b) and (c). Upon completion of these steps, the quantity of the detected target nucleic acid is divided by the quantity of the collected feces. By so doing, the variance in the measurement result due to the variance in the quantity of the collected feces can be corrected. Hence, highly reliable detection results with reduced influence of inhibitory substances can be obtained similarly to with the case where a fixed quantity of feces has been collected in advance.

In addition, if the quantity of the collected feces varies, it is possible to correct the variance in the quantity of the collected feces by preparing a nucleic acid solution of a volume proportional to the quantity of the collected feces, at the time when preparing the nucleic acid solution after the recovery of the nucleic acids eluted in the extraction solution. Here, the term “volume proportional to the quantity of feces” means that the volume of the nucleic acid solution prepared by recovering nucleic acids from a unit quantity of feces is fixed. For example, in a case where the quantity of the collected feces of three specimens varies, respectively, at 1 g, 1.5 g, and 2 g, and in a case where the nucleic acids recovered from the specimen whose quantity of feces is 1 g have been prepared into 100 μL of a nucleic acid solution; then, the nucleic acids recovered from the specimen whose quantity of feces is 1.5 g are to be prepared into 150 μL (1.5×100 μL) of a nucleic acid solution; and the nucleic acids recovered from the specimen whose quantity of feces is 2.0 g are to be prepared into 200 μL (2×100 μL) of a nucleic acid solution.

Furthermore, it is also possible that: after the liquid quantity of the extraction solution to be added to the collected feces or the solid content thereof has been set at a volume proportional to the quantity of the collected feces, the nucleic acid solution is prepared at a volume proportional to the quantity of the collected feces, at the time when preparing the nucleic acid solution after the recovery of the nucleic acids eluted in the extraction solution. By so doing, the variance in the quantity of inhibitory substances carried over into the nucleic acids dependent on the variance in the quantity of the collected feces can be more effectively reduced.

In addition, it is also possible that: after the liquid quantity of the extraction solution to be added to the collected feces or the solid content thereof has been set at a volume proportional to the quantity of the collected feces, a fixed volume of an aliquot is dispensed from the extraction solution, and then a fixed volume of a nucleic acid solution is prepared by recovering the nucleic acids in this dispensed solution. By so doing, the variance in the quantity of inhibitory substances carried over into the finally recovered nucleic acid solution can be reduced.

By preparing a plurality of fecal specimens in such a manner, the quantities of nucleic acids and inhibitory substances contained in a fixed volume of the yielded nucleic acid solution of any specimen can be equivalent to the quantities of nucleic acids and inhibitory substances contained in a fixed quantity of feces. For this reason, by using a fixed volume of an aliquot dispensed from each nucleic acid solution for the reaction to detect a nucleic acid, the difference in the quantity of inhibitory substances carried over into the respective reaction systems between specimens, can be suppressed within the level of individual difference of the concentration of inhibitory substances contained in feces. Hence, highly reliable detection results with reduced influence of inhibitory substances can be obtained similarly to with the case where a fixed quantity of feces has been collected in advance.

The method for detecting a target nucleic acid is not particularly limited, and can be conducted by using any known means for use in the detection and analysis of a specific nucleic acid. Examples thereof include a method for detecting a specific nucleotide sequence region by analyzing an amplicon through PCR or such a nucleic acid amplification reaction. In addition, when it comes to the RNA recovery, it is possible to synthesize cDNA from the total RNA recovered from feces through a reverse transcription reaction, and then analyze the synthesized cDNA in the same manner as that of the DNA analysis.

The target nucleic acid of the present invention is not specifically limited as long as it is a nucleic acid serving as an analysis object of detection, quantification, or the like, and as long as its nucleotide sequence has been elucidated to a detectable degree by PCR or such a usual technique for the analysis of a nucleic acid. Examples thereof include DNA and mRNA derived from animals. It is preferable that the target nucleic acid is RNA such as mRNA. In addition, in the present invention, there is no particular limitation as long as it is a nucleic acid derived from an animal which excretes feces, although preferred is a nucleic acid derived from a mammalian cell, and more preferred is a nucleic acid derived from a human.

For example, by adopting an appropriate target nucleic acid, it is possible to detect the presence or absence of a genetic variation, such as a nucleotide sequence region in which a cancer gene or the like is encoded or a nucleotide sequence region including microsatellites. By so doing, the presence or absence of the onset of cancer can be examined. When using DNA recovered from a fecal sample, for example, it is possible to detect a DNA mutation such methylation, nucleotide insertion, deletion, substitution, duplication, or inversion. In addition, when using recovered RNA, for example, it is possible to detect an RNA mutation, such as nucleotide insertion, deletion, substitution, duplication, inversion, or splicing variant (isoform). Moreover, the RNA expression level can also be detected. It is particularly preferable to conduct an mRNA expression analysis, a mutation analysis of the K-ras gene, an analysis of DNA methylation, or the like. Note that these analyses can be carried out by using known methods in the art. Moreover, it is also possible to use a commercially available analysis kit such as a K-ras gene mutation analysis kit and a methylation detection kit.

In the present invention, it is preferable to adopt, as the target nucleic acid, a nucleic acid derived from a cell of a digestive tract such as the large intestine, small intestine, and stomach, because the nucleic acid is to be recovered from feces. It is more preferable to adopt, as the target nucleic acid, a nucleic acid derived from an exfoliated cell from the large intestine.

It is particularly preferable to adopt, as the target nucleic acid, a nucleic acid derived from a marker gene of neoplastic transformation (including canceration) or a marker gene of an inflammatory digestive organ disease. It is more preferable to adopt, as the target nucleic acid, a nucleic acid derived from a marker gene of colon cancer. The term “nucleic acid derived from a gene” means genomic DNA, or an expression product such as mRNA, of the gene. Examples of the marker which indicates neoplastic transformation can include known cancer markers such as the COX-2 (cyclooxygenase-2) gene, the carcinoembryonic antigen (CEA), and the sialyl Tn (STN) antigen, and the presence or absence of mutation(s) in the APC gene, the p53 gene, the K-ras gene, and the like. Moreover, the detection of methylation of p16, hMLHI, MGMT, p14, APC, E-cadherin, ESR1, SFRP2, or such a gene is also useful as a diagnosis marker for colonic diseases (for example, refer to Lind et al., “A CpG island hypermethylation profile of primary colorectal carcinomas and colon cancer cell lines”, Molecular Cancer, 2004, Vol. 3, Chapter 28). On the other hand, examples of the marker which indicates an inflammatory digestive organ disease can include a nucleic acid derived from the COX-2 gene and the like.

By adopting such a nucleic acid derived from a marker gene of a specific disease as the target nucleic acid, and detecting it by using the detection method of the present invention, it is possible to examine the presence or absence of the affection and the stage of the advancement of a disease such as cancer, inflammatory disease, or the like. For example, it is possible to determine whether or not the examinee is affected by the disease, by previously setting a threshold regarding the quantity of the target nucleic acid in feces, and checking the quantity of the target nucleic acid in the specimen detected by using the detection method of the present invention, on the basis of this threshold. The threshold for use in this occasion can be appropriately set, for example, by conducting the detection method of the present invention to obtain the quantity of the target nucleic acid in feces collected from a group of subjects who have been proven to be not affected by the diseases, and the quantity of the target nucleic acid in feces collected from a group of subjects who have been proven to be affected by the diseases, and then making a comparison between these measurement values of both groups.

For example, collected feces are subjected to the detection method of the present invention by adopting a nucleic acid derived from a marker gene of colon cancer whose expression level increases (including a case where its expression is induced) in colon cancer patients, such as a nucleic acid derived from the COX-2 gene, as the target gene. Then, the quantity of the detected target nucleic acid is compared with a preset threshold. When the quantity of the target nucleic acid is equal to or greater than the threshold, the determination can be made that the person who provided the feces is affected by colon cancer. Conversely, when the quantity of the target nucleic acid is smaller than the threshold, the determination can be made that the person who provided the feces is not affected by colon cancer. It is also possible to adopt, as the target nucleic acid, a kind of marker gene-derived nucleic acid whose expression level decreases in colon cancer patients so that the determination can be made that the examinee is affected by colon cancer when the quantity of the detected target nucleic acids is equal to or smaller than a preset threshold, and that the examinee is not affected by colon cancer when the quantity is greater than the threshold.

### EXAMPLES

Next is a more detailed description of the present invention with reference to Examples. However, the present invention is not to be limited to the Examples below. The MKN45 cells had been cultured by a usual method before use.

### Example 1

### Application to the Expression Analysis of Colon Cancer-Related Gene: 1

Feces of a healthy subject was mixed well to be homogenized, which was then added with MKN45 cells so that 1×10⁵ cells could be contained per gram of the fecal sample. This product was then mixed. Although MKN45 cells are derived from stomach cancer, they abundantly express the COX-2 gene similarly to colon cancer cells. Therefore, the thus mixed fecal sample was used as an artificial sample imitating feces collected from a colon cancer patient.

From the mixed fecal sample, 1 cm³ was respectively measured out per sample. In total, six samples were prepared. These were respectively placed in a 15 mL centrifugal tube (a product of Falcon) and preserved at 4° C. until the next step. The fecal samples were collected by using the sampling rod 13, that is, a sampling jig as illustrated in FIG. 1 (the sampling rod 13 integrally unified with the cap 12). The sampling rod 13 comprises a hole 13a capable of collecting 1 cm³ of feces and a movable lid 13b capable of enclosing it.

Thereafter, in each tube, 3 mL of an extraction solution (acid phenol guanidine solution) was added and suspended. Then, the suspension was centrifuged at 12,000×g at 4° C. for 20 minutes. The supernatant (aqueous layer) yielded from the centrifugal separation was passed through the RNA recovery column of the RNeasy midi kit (a product of Qiagen GmbH), followed by the process to wash the RNA recovery column and the process to elute RNA according to the appended protocol. By so doing, RNA was recovered in the form of 50 μL of an RNA solution.

The quantity of the recovered total RNA was determined by measuring the concentration of each RNA solution using a NanoDrop instrument (a product of NanoDrop Technologies, Inc). The measurement results of the RNA concentrations of the respective RNA solutions are shown in Table 1. The RNA solutions of these six samples were expected to show a comparable level of concentration because they had been extracted from feces of the same fixed volume. However, in fact, the results showed a variance between samples.

TABLE 1

| Sample No. | RNA concentration (ng/μL) |
|---|---|
| 1 | 533 |
| 2 | 109 |
| 3 | 348 |
| 4 | 375 |
| 5 | 210 |
| 6 | 144 |

The cDNA was synthesized from the RNA recovered in the RNA solution, by using a commercially available reverse transcription reaction kit (a product of Invitrogen). At this time, the synthesis was carried out under two types of reaction conditions regarding the RNA quantity for use in the reverse transcription reaction as follows: (a) a condition in which 1 μL of the RNA solution was added to the reaction solution of the reverse transcription reaction, irrespective of the concentration of the recovered RNA solution; and (b) a condition in which the quantity of the RNA solution to be added to the reaction solution was adjusted in accordance with the concentration of the RNA solution so that 1 μg of RNA could be added to the reaction solution of each reverse transcription reaction.

The expression product (mRNA) of the COX-2 gene was detected by real-time PCR with use of the obtained cDNA as a template. The real-time PCR primers were those of the COX-2 primer probe MIX (catalog No: Hs00153133_m1) manufactured by Applied Biosystems, Inc. Specifically speaking, 1 μL of the respective cDNA was dispensed in a 0.2 mL 96-well PCR plate. Then, in each well, 8 μL of ultrapure water, 10 μL of a nucleic acid amplifying reagent (TaqMan Gene Expression Master Mix, a product of Applied Biosystems, Inc.), and 1 μL of the COX-2 primer probe MIX (a product of Applied Biosystems, Inc) were added and mixed respectively. By so doing, the PCR reaction solution was prepared. The PCR plate was set in the ABI real-time PCR system, where PCR was carried out through a heat treatment at 95° C. for 10 minutes, then 40 heat cycles, each cycle consisting of 95° C. for 1 minute, 56.5° C. for 1 minute, and 72° C. for 1 minute, and a final heat treatment at 72° C. for 7 minutes, while measuring the fluorescence intensity in a time course manner.

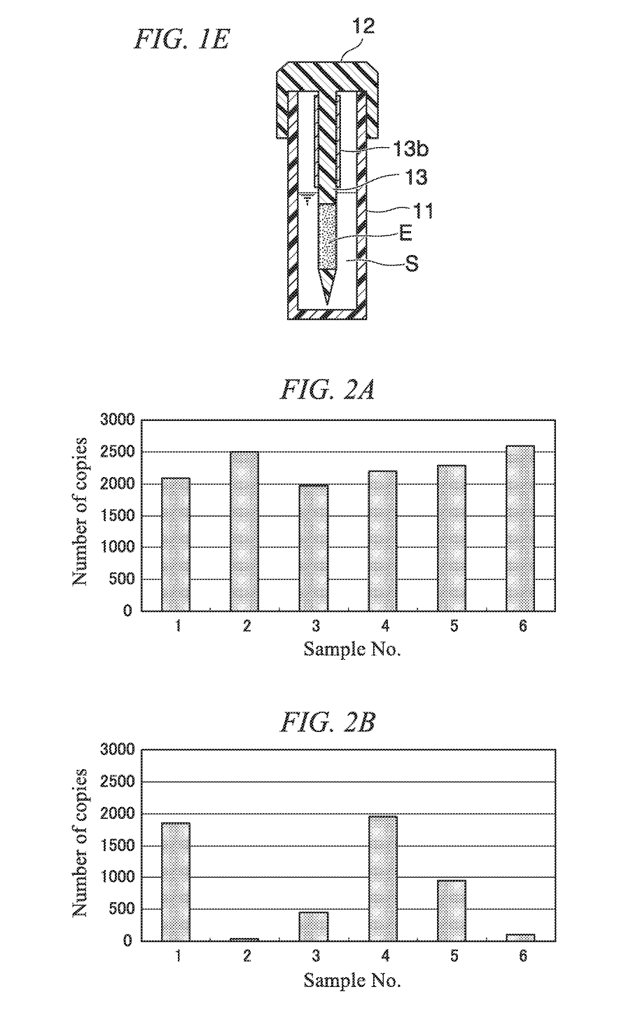

The measurement results of the fluorescence intensity were analyzed to calculate the expression levels of the COX-2 gene in the RNA recovered from the respective samples, which are shown in the graphs of FIG. 2A to FIG. 2B. FIG. 2A shows the results of the case where the RNA quantity for use in the reverse transcription reaction followed the condition (a), while FIG. 2B shows the results of the case where the RNA quantity followed the condition (b). As a result, it was found that the difference between samples, that is, the variance was smaller in the condition (a) than in the condition (b). In the first place, these six samples had been prepared from the same fecal sample through RNA recovery. Hence, in theory, the quantities of the expression product of the COX-2 gene contained in each sample should be substantially equal to each other. In other words, seeing these results, the method adopting the condition (a) in which a fixed volume of the RNA solution was added to the reverse transcription reaction, irrespective of the concentration of the recovered RNA, namely the detection method of the present invention, contributed to more accurate results and was proven to be more suitable for the detection of the expression of a gene, than the method adopting the condition (b) in which the RNA quantity to be added to the reaction solution was set equal.

The reason why the variation between samples was greater in the condition (b) even though the RNA quantity added to the reaction solution was set equal, is thought to be that: since the quantity of the added RNA solution varied depending on the sample, the quantity of inhibitory substances carried over into the reaction solution also varied, which interfered with accurate measurement of the expression level. In addition, most RNA contained in feces are derived from bacteria. Therefore, even though the RNA concentration has been measured and an RNA quantity equivalent to 1 μg was added, the quantity of a human-derived nucleic acid serving as the target can not be always fixed. This can also be suggested as a reason why it difficult to obtain accurate detection results.

### Example 2

### Application to the Expression Analysis of Colon Cancer-Related Gene: 2

Feces of a healthy subject was mixed well to be homogenized, 1 g of which was respectively weighed out per sample. In total, six samples were prepared. Five samples of these were respectively added with 1×10², 1×10³, 1×10⁴, 1×10⁵, and 1×10⁶ MKN45 cells and mixed well respectively. The remaining one sample was not added with MKN45 cells. These six samples were respectively suspended in 5 mL of a 70% ethanol solution filled in a 15 mL centrifugal tube (a product of Falcon).

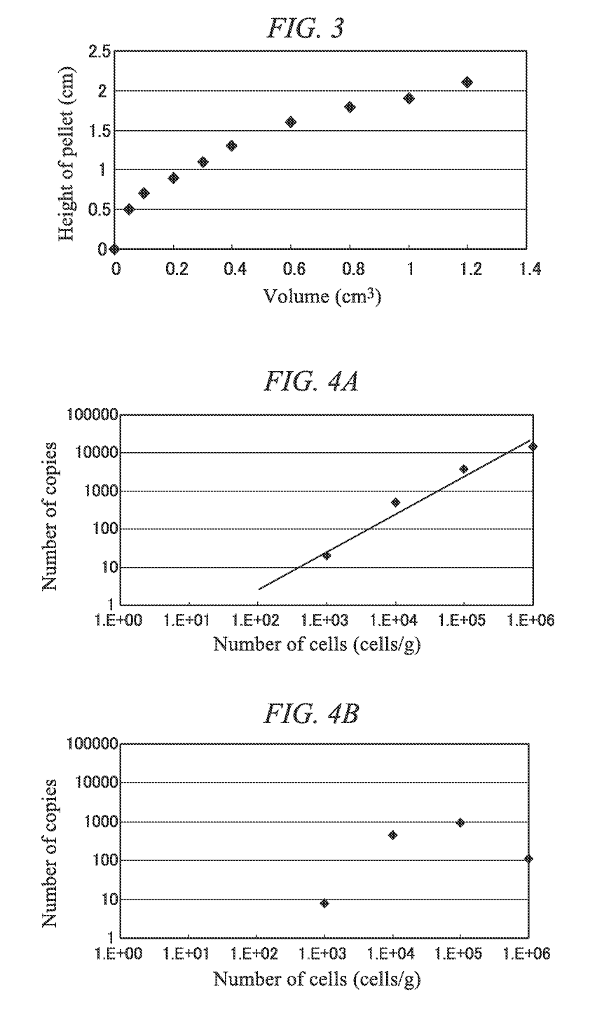

The volume of the feces-origin solid content of each sample was measured by two types of measurement methods: the absorbance, and the height of a pellet of precipitated feces. Specifically speaking, the obtained suspension was left still at 25° C. for one day, and the absorbance of the supernatant thereof was measured with a wavelength of 450 nm. Then, this was centrifuged at 2,000×g for 10 minutes, and the height of the pellet of the precipitated feces was measured by the Smart sensor (OMRON Corporation). FIG. 3 is a correlation graph between the height of the pellet and the volume of the solid content, which had been obtained in advance using feces having a known volume of the solid content by preparing an ethanol suspension thereof, subjecting it to a centrifugal treatment, and measuring the height of the pellet in the same manner. Using this correlation graph, the volume of the solid content of each sample was estimated from the measured height of each pellet.

In order to recover RNA from each sample, the supernatant was removed and the remaining solid content was added with 3 mL of an extraction solution (acid phenol guanidine solution) and suspended. Then, in the same manner as that of Example 1, RNA was recovered in the form of 50 μl, of an RNA solution, and the quantity of the recovered total RNA was determined by measuring the concentration of each RNA solution.

Table 2 shows the content of MKN45 cells, the absorbance, the estimated volume of the solid content, and the RNA concentration of the RNA solution of the respective samples. As a result, the difference in the RNA concentration was found to be large between samples. Here, since most RNAs contained in feces are derived from bacteria, it is considered that the content of MKN45 cells has almost no influence on the recovered RNA quantity. Hence, possibly, the difference in the RNA concentration between samples could be the reflection of the variance in the abundance of bacteria in fecal samples.

TABLE 2

| | Content of | Weight | | Estimated | |
|---|---|---|---|---|---|
| | MKN45 | of | | volume of | RNA |
| Sample | cells | feces | Absor- | solid content | concentration |
| No. | (cells/g) | (g) | bance | (cm³) | (ng/μL) |
| 1 | 0 (not | 1.0 | 0.12 | 0.7 | 263 |
| | added) | | | | |
| 2 | 10² | 1.0 | 0.11 | 0.7 | 401 |
| 3 | 10³ | 1.0 | 0.11 | 0.6 | 149 |
| 4 | 10⁴ | 1.0 | 0.11 | 0.7 | 520 |
| 5 | 10⁵ | 1.0 | 0.12 | 0.7 | 188 |
| 6 | 10⁶ | 1.0 | 0.11 | 0.7 | 127 |

The cDNA was synthesized from the RNA recovered in the RNA solution, by using a commercially available reverse transcription reaction kit (a product of Invitrogen). At this time, similarly to with Example 1, the synthesis was carried out under two types of reaction conditions regarding the RNA quantity for use in the reverse transcription reaction as follows: (a) a condition in which 1 μL of the RNA solution was added to the reaction solution of the reverse transcription reaction, irrespective of the concentration of the recovered RNA solution; and (b) a condition in which the quantity of the RNA solution to be added to the reaction solution was adjusted in accordance with the concentration of the RNA solution so that 1 μg of RNA could be added to the reaction solution of each reverse transcription reaction.

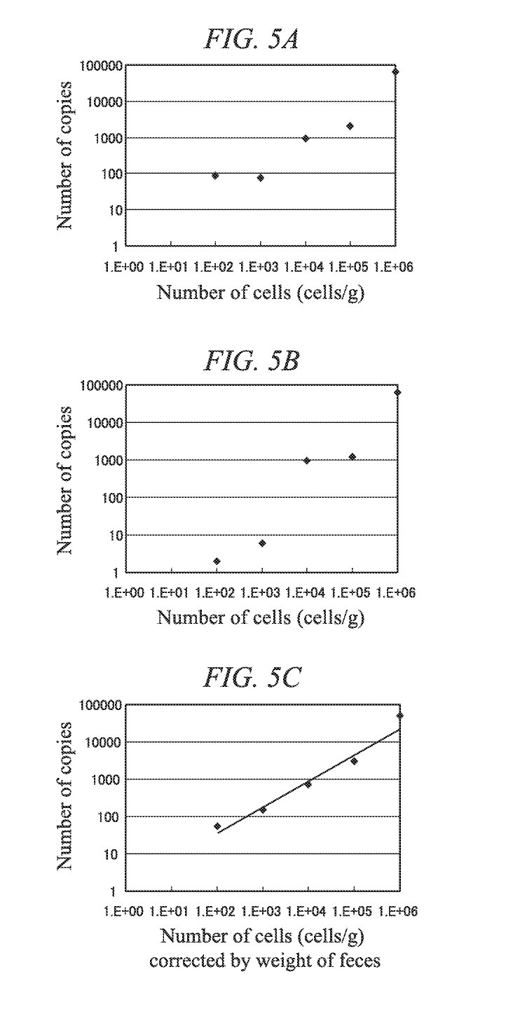

Thereafter, real-time PCR was carried out with use of the obtained cDNA as a template in the same protocol as that of Example 1. The measurement results of the fluorescence intensity were analyzed to calculate the expression levels of the COX-2 gene in the RNA recovered from the respective samples, which are shown in the graphs of FIG. 4A and FIG. 4B. FIG. 4A shows the results of the case where the RNA quantity for use in the reverse transcription reaction followed the condition (a); while FIG. 4B shows the results of the case where the RNA quantity followed the condition (b). Note that the straight line in FIG. 4A is a linear approximation obtained from the respective measurement values. As a result, as shown in FIG. 4A, the expression level of the COX-2 gene was higher as the content of MKN45 cells added to feces increased, showing correlated results, in the case of the condition (a) to check the COX-2 expression level, in which 1 μL of the RNA solution was used for the reverse transcription reaction, irrespective of the RNA concentration. On the other hand, no correlation was found between the content of MKN45 cells and the number of copies of the COX-2 gene in the case of the condition (b) in which an RNA quantity equivalent to 1 μg was added to the reverse transcription reaction. The reason is thought to be that: since the quantity of the RNA solution added to the solution of the reverse transcription reaction varied depending on the sample, the quantity of inhibitory substances carried over thereinto also varied, which made the difference in the influence of inhibitory substances between samples. In addition, in order to add RNA to be equivalent to 1 μg, it is necessary to add 2 μL or more of the RNA solution to the solution of the reverse transcription reaction regarding almost all samples. For this reason, a large quantity of feces-origin reaction inhibitory substances contained in the extracted RNA might have been carried over into the reaction solution. This can also be suggested as a possible reason for the adverse effect on the results.

In other words, these results showed that, similarly to with Example 1, since the quantity of the RNA solution supplied to the reaction to detect a nucleic acid varied depending on the sample, the quantity of feces-origin inhibitory substances carried over into the reaction solution also varied, which interfered with accurate measurement of the gene expression level in feces, and the use of the detection method of the present invention contributed to more accurate results and was proven to be suitable for the detection of the expression of a gene.

### Example 3

### Application to the Expression Analysis of Colon Cancer-Related Gene: 3

Feces of a healthy subject was mixed well to be homogenized, roughly about 1 g of which was respectively weighed out per sample. In total, six samples were prepared. Five samples of these were respectively added with 1×10², 1×10³, 1×10⁴, 1×10⁵, and 1×10⁶ MKN45 cells and mixed well respectively. The remaining one sample was not added with MKN45 cells.

These six samples were respectively weighed, and suspended in 5 mL of a 70% ethanol solution similarly to with Example 2. Then, the absorbance and the height of a pellet were measured. With use of the correlation graph of FIG. 3, the volume of the solid content of each sample was estimated. Thereafter, additionally, in the same manner as that of Example 2, RNA was recovered from each sample in the form of 50 μL of an RNA solution, and the quantity of the recovered total RNA was determined by measuring the concentration of each RNA solution. Table 3 shows the content of MKN45 cells, the weight, the absorbance, the estimated volume of the solid content, and the RNA concentration of the RNA solution of the respective samples.

TABLE 3

| | Content of | Weight | | Estimated | |
|---|---|---|---|---|---|
| | MKN45 | of | | volume of | RNA |
| Sample | cells | feces | Absor- | solid content | concentration |
| No. | (cells/g) | (g) | bance | (cm³) | (ng/μL) |
| 1 | 0 (not | 1.4 | 0.16 | 1.0 | 392 |
| | added) | | | | |
| 2 | 10² | 1.6 | 0.18 | 1.1 | 249 |
| 3 | 10³ | 0.5 | 0.05 | 0.3 | 135 |
| 4 | 10⁴ | 1.3 | 0.14 | 0.9 | 430 |
| 5 | 10⁵ | 0.7 | 0.08 | 0.6 | 338 |
| 6 | 10⁶ | 1.3 | 0.14 | 0.9 | 487 |

The cDNA was synthesized from the RNA recovered in the RNA solution, by using a commercially available reverse transcription reaction kit (a product of Invitrogen). At this time, similarly to with Example 1, the synthesis was carried out under two types of reaction conditions regarding the RNA quantity for use in the reverse transcription reaction as follows: (a) a condition in which 1 μL of the RNA solution was added to the reaction solution of the reverse transcription reaction, irrespective of the concentration of the recovered RNA solution; and (b) a condition in which the quantity of the RNA solution to be added to the reaction solution was adjusted in accordance with the concentration of the RNA solution so that 1 μg of RNA could be added to the reaction solution of each reverse transcription reaction.

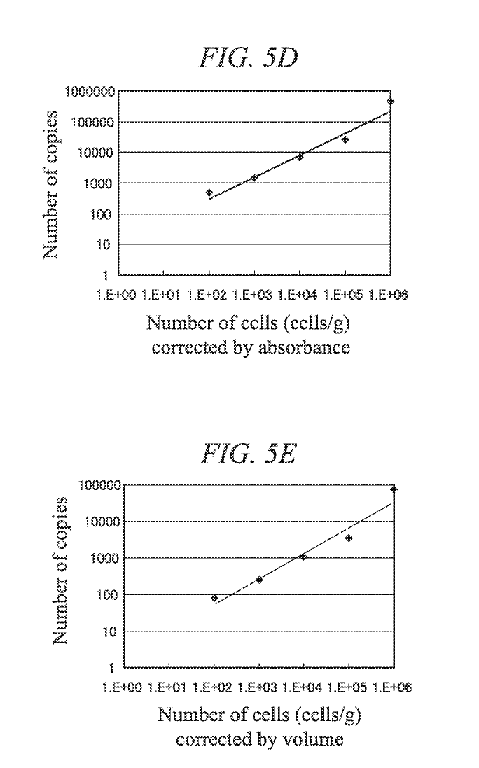

Thereafter, real-time PCR was carried out with use of the obtained cDNA as a template in the same protocol as that of Example 1. The measurement results of the fluorescence intensity were analyzed to calculate the expression levels of the COX-2 gene in the RNA recovered from the respective samples, which are shown in the graphs of FIG. 5A to FIG. 5E. FIG. 5A shows the results of the case where the RNA quantity for use in the reverse transcription reaction followed the condition (a), while FIG. 5B shows the results of the case where the RNA quantity followed the condition (b). As a result, similarly to with Examples 1 and 2, no correlation was found between the content of MKN45 cells and the number of copies of the COX-2 gene in the case of the condition (b). On the other hand, in the case of the condition (a), a linearity was found between the content of MKN45 cells and the number of copies of the COX-2 gene as compared to the case of the condition (b). However, because the feces had been collected by roughly weighing its quantity, the quantity of feces had a variance. Thus, the calculated expression level (number of copies) of the COX-2 gene showed a variance.

Therefore, the expression level calculated from the real-time PCR was corrected by the quantity of feces. FIG. 5C shows the expression level obtained from the correction of the expression level as shown in FIG. 5A by the quantity (weight) of feces of Table 3. FIG. 5D shows the expression level obtained from the correction of the expression level as shown in FIG. 5A by the quantity (absorbance) of feces of Table 3. FIG. 5E shows the expression level obtained from the correction of the expression level as shown in FIG. 5A by the quantity (estimated volume of the solid content) of feces of Table 3. Note that in this Example, the term “corrected by the quantity of feces” means that the expression level is divided by the respective quantity of feces. Moreover, the straight line in FIGS. 5C to E is a linear approximation obtained from the respective measurement values.

As a result, as is apparent from FIGS. 5C to E, respectively, strong correlations were found between the content of MKN45 cells and the number of copies of the COX-2 gene.

From these results, it is apparently possible, even though the initial quantity of feces (the quantity of collected feces) varies, to reduce the influence of inhibitory substances by adjusting the quantity of the RNA solution to be added to the reverse transcription reaction at a fixed value, and to more accurately detect the expression level by correcting the thus obtained results by the quantity of feces.

### Example 4

### GAPDH Detection Using Clinical Specimens

20 g of feces were respectively collected from five colon cancer patients, and respectively mixed well to be homogenized. These were respectively dispensed at 0.5 cc. By so doing, five fecal samples (C1 to C5) were prepared. In addition, feces of five healthy subjects were also respectively dispensed at 0.5 cc in the same manner, thereby preparing five fecal samples (N1 to N5).

RNA was recovered from each of these samples. Specifically speaking, 3 mL of a phenol mixture “Trizol” (a product of Invitrogen Corporation) was added to each sample, and sufficiently mixed for 30 seconds or longer by using a homogenizer. Then, 3 mL of chloroform was added thereto. This was again sufficiently mixed by vortexing, and then centrifuged at 12,000×g at 4° C. for 20 minutes. The supernatant (aqueous layer) yielded from the centrifugal separation was passed through the RNA recovery column of the RNeasy midi kit (a product of Qiagen GmbH), followed by the process to wash the RNA recovery column and the process to elute RNA according to the appended protocol. By so doing, RNA was recovered in the form of 50 μL of an RNA solution. The quantity of the recovered total RNA was determined by measuring the concentration of each RNA solution using the NanoDrop instrument (a product of NanoDrop Technologies, Inc).

The cDNA was synthesized from the RNA recovered in the RNA solution, by using a commercially available reverse transcription reaction kit (a product of Invitrogen). At this time, the synthesis was carried out under two types of reaction conditions regarding the RNA quantity for use in the reverse transcription reaction as follows: (a) a condition in which 0.5 μL or 0.25 μL of the RNA solution was added to the reaction solution of the reverse transcription reaction, irrespective of the concentration of the recovered RNA solution; and (b) a condition in which the quantity of the RNA solution to be added to the reaction solution was adjusted in accordance with the concentration of the RNA solution so that 1 μg, 0.5 μg, 0.25 μg, or 0.125 μg of RNA could be added to the reaction solution of each reverse transcription reaction.

Thereafter, the expression level of the human GAPDH (glyceraldehyde 3-phosphate dehydrogenase) was measured by PCR with use of the obtained cDNA as a template. Specifically speaking, the cDNA was added with 12.5 μL of the 2× TaqMan PCR master mix (a product of Applied Biosystems, Inc) and a primer probe set for human GAPDH detection (a product of Applied Biosystems, Inc) so that the final volume was set to 25 μL. By so doing, a PCR solution was prepared. The PCR solution was subjected to TaqMan PCR analysis by using the ABI Prism 7700 Sequence Detection System (a product of Applied Biosystems, Inc). The heat cycle of PCR was in accordance with the usage instruction. The quantification was conducted on the basis of the results of the fluorescence intensity obtained by using a dilution series having known concentrations of a standard plasmid as a template.

Table 4 shows the measurement results of the GAPDH gene expression levels with respective quantities [volumes] of RNA solutions used for the reverse transcription reaction, in the case where the RNA quantity for use in the reverse transcription reaction followed the condition (a). In Table 4, the term “RNA weight” means the weight of RNA added to the reaction solution of the reverse transcription reaction. As a result, a dilution linearity of the GAPDH gene expression level was seen in all specimens in the case where the quantity of the RNA solution used for the reverse transcription reaction was between 0.25 and 0.5 μL. Therefore, it was found that there was no inhibitory effect of feces-origin inhibitory substances at least when 0.5 μL or smaller quantity of RNA solution had been added.

TABLE 4

| Quantity of RNA solution added to reaction | | 0.5 μL | | 0.25 μL | |
|---|---|---|---|---|---|
| solution of reverse transcription reaction | | | GAPDH | | GAPDH |
| | RNA | RNA | expression | RNA | expression |
| Sample | concentration | weight | level (number | weight | level (number |
| No. | (ng/μL) | (ng) | of copies) | (ng) | of copies) |
| C1 | 210 | 105 | 2143 | 53 | 1109 |
| C2 | 562 | 281 | 206 | 141 | 102 |
| C3 | 285 | 143 | 230 | 71 | 121 |
| C4 | 135 | 68 | 1607 | 34 | 812 |
| C5 | 336 | 168 | 633 | 84 | 317 |
| N1 | 326 | 163 | 1801 | 82 | 896 |
| N2 | 143 | 72 | 875 | 36 | 451 |
| N3 | 450 | 225 | 524 | 113 | 276 |
| N4 | 625 | 313 | 887 | 156 | 445 |
| N5 | 228 | 114 | 229 | 57 | 113 |

TABLE 5

| | | Quantity of RNA added to reaction solution of reverse transcription reaction | | | | | | | |
|---|---|---|---|---|---|---|---|---|---|
| | | 1 μg | | 0.5 μg | | 0.25 μg | | 0.125 μg | |
| | | | GAPDH | | GAPDH | | GAPDH | | GAPDH |
| | | Volume | expression | Volume | expression | Volume | expression | Volume | expression |
| | RNA | of | level | of | level | of | level | of | level |
| | concentration | addition | (number | addition | (number | addition | (number | addition | (number |
| Sample No. | (ng/μL) | (μL) | of copies) | (μL) | of copies) | (μL) | of copies) | (μL) | of copies) |
| C1 | 210 | 4.8 | 0 | 2.4 | 512 | 1.2 | 2312 | 0.6 | 2219 |
| C2 | 562 | 1.8 | 1 | 0.9 | 12 | 0.4 | 165 | 0.2 | 88 |
| C3 | 285 | 3.5 | 0 | 1.8 | 0 | 0.9 | 11 | 0.4 | 189 |
| C4 | 135 | 7.4 | 0 | 3.7 | 0 | 1.9 | 20 | 0.9 | 37 |
| C5 | 336 | 3.0 | 2312 | 1.5 | 1680 | 0.7 | 887 | 0.4 | 420 |
| N1 | 326 | 3.1 | 1230 | 1.5 | 5485 | 0.8 | 2879 | 0.4 | 1440 |
| N2 | 143 | 7.0 | 0 | 3.5 | 0 | 1.7 | 12 | 0.9 | 23 |
| N3 | 450 | 2.2 | 2340 | 1.1 | 1207 | 0.6 | 621 | 0.3 | 292 |
| N4 | 625 | 1.6 | 3019 | 0.8 | 1476 | 0.4 | 710 | 0.2 | 367 |
| N5 | 228 | 4.4 | 267 | 2.2 | 864 | 1.1 | 459 | 0.5 | 228 |

On the other hand, Table 5 shows the measurement results of the GAPDH gene expression levels with respective RNA quantities used for the reverse transcription reaction, in the case where the RNA quantity for use in the reverse transcription reaction followed the condition (b). In Table 5, the term “Volume of addition” means the quantity of the RNA solution added to the reaction solution of the reverse transcription reaction. The dilution linearity was examined with respect to these results by making comparisons between the cases where the RNA quantity added to the reaction solution of the reverse transcription reaction was 1 μg and 0.5 μg, and between the cases of 0.5 μg and 0.25 μg, respectively.

As a result, among the colon cancer specimens (samples C1 to C5), only the sample C5 between 0.25 μg and 0.5 μg, and only the samples C5 and C2 between 0.125 μg and 0.25 μg showed the dilution linearity of the GAPDH gene expression level relative to the RNA quantity. From these results, only the cases where the samples C5 and C2 were added so that the quantity of RNA addition was set to be 0.125 μg can be said to receive no influence from feces-origin inhibitory substances on the reverse transcription reaction and the subsequent PCR, and the other data were influenced by inhibitory substances. Thus, the reliability of these data was found to be inadequate.

Considering that the samples C2 and C5 which showed relatively good dilution linearity were relatively high in the concentration of the RNA solution recovered from feces, among the colon cancer specimens, and also considering the results of Table 4, the reason why the dilution linearity was not seen in the colon cancer specimens as presented in Table 5 is thought to be that the quantity of the addition of the RNA solution was too large at 0.5 μL or more. Here, the difference in the recovered RNA quantity was mainly due to the individual difference in the quantity of bacteria in feces. Thus, it was found to be necessary to take into account both factors of the individual difference in the concentration of inhibitory substances in feces and the individual difference in the concentration of the recovered RNA (bacterial RNA) in feces, when it comes to the case where RNA is to be added to the reaction system of the reaction to detect a nucleic acid on the weight basis, and it was also found to be necessary for eliminating the influence of these inhibitory substances to increase the dilution fold (meaning to adequately reduce the quantity to be added to the reaction solution of the reaction to detect a nucleic acid). In particular, in this Example, there was almost no influence of inhibitory substances carried over from feces when 0.25 μL of the recovered RNA solution, as an aliquot out of 50 μL, was added to 20 μL volume of the solution of the reverse transcription reaction. By considering this result, it is preferable to add a recovered RNA solution to the reaction to detect a nucleic acid so that the dilution would be about 80-fold (20 μL/0.25 μL), when it comes to the case where RNA is recovered from feces in a similar scale to that of this Example.

On the other hand, the dilution linearity was also examined on the healthy subject specimens (samples N1 to N5) in the same manner. Only the sample N2 did not show the dilution linearity between 0.25 μg and 0.5 μg, whereas all the other samples showed the dilution linearity.

In this way, the results showed a difference in the influence of inhibitory substances between cancer patients and healthy subjects, meaning that colon cancer specimens are more susceptible to inhibitory substances than healthy subject specimens. This can be attributed to the assumption that the quantity of inhibitory substances contained in feces is greater in colon cancer patients than in healthy subjects. In particular, in the cases of specimens having low RNA concentration in the recovered RNA solution, the volume of addition of the RNA solution to the reaction solution of the reverse transcription reaction increases, and thus the quantity of inhibitory substances carried over thereinto also increases. Because of this reason, the influence thereof of inhibitory substances becomes prominent.

In cases where RNA is recovered from samples having a same initial quantity by a same recovery method, the concentration of inhibitory substances existing in the recovered RNA solution would vary due to the individual difference. For this reason, it is necessary to adjust the quantity of addition of the RNA solution so that the influence of inhibitory substances can be avoided. However, like the detection method of the present invention, it is readily possible, by determining the RNA [quantity] to be brought into the reaction to detect a nucleic acid on the volume basis, to set an appropriate condition where such an influence of inhibitory substances can be eliminated.

### Example 5

### COX-2 Detection Using Clinical Specimens

The expression level of the human COX-2, the expression of which is specifically found in feces of cancer patients, was measured by PCR with use of the cDNA synthesized in Example 4 as a template. Specifically speaking, TaqMan PCR analysis was carried out in the same manner as that of Example 4 except for using a primer probe set for human COX-2 gene detection (a product of Applied Biosystems, Inc) instead of using the primer probe set for human GAPDH detection (a product of Applied Biosystems, Inc).

Table 6 shows the measurement results of the COX-2 gene expression levels with respective quantities [volumes] of RNA solutions used for the reverse transcription reaction, in the case where the RNA quantity for use in the reverse transcription reaction followed the condition (a). In Table 6, the term “RNA weight” means the same as that of Table 4. As a result, it was possible to conduct the detection within a range where no inhibition would be effective, departing from a range of influence of inhibition effect expected from the individual difference.

On the other hand, Table 7 shows the measurement results of the COX-2 gene expression levels with respective RNA quantities used for the reverse transcription reaction, in the case where the RNA quantity for use in the reverse transcription reaction followed the condition (b). In Table 7, the term “Volume of addition” means the same as that of Table 5. In the results of the colon cancer specimens (samples C1 to C5), mixedly, some specimens showed lowered signals due to the influence of inhibition even though the quantity of RNA addition was 0.125 μg, while other specimens showed lowered signals due to the dilution (in other words, the quantity of RNA addition was too small). In short, the case of the condition (b) was proven to be inferior in the reliability of the obtained detection results to the case of the condition (a).

In the healthy subject specimens (N1 to N5), almost no expression of COX-2 was seen from the beginning. So, it was assumed that the influence of inhibitory substances would be little even though RNA was added on the weight basis. For this reason, it was considered that almost no influence would be imposed on the specificity in the case of the detection of the COX-2 expression level.

TABLE 6

| Quantity of RNA solution added to reaction | | 0.5 μL | | 0.25 μL | |
|---|---|---|---|---|---|
| solution of reverse transcription reaction | | | COX-2 | | COX-2 |
| | RNA | RNA | expression | RNA | expression |
| Sample | concentration | weight | level (number | weight | level (number |
| No. | (ng/μL) | (ng) | of copies) | (ng) | of copies) |
| C1 | 210 | 105 | 189 | 53 | 89 |
| C2 | 562 | 281 | 772 | 141 | 396 |
| C3 | 285 | 143 | 19 | 71 | 11 |
| C4 | 135 | 68 | 210 | 34 | 98 |
| C5 | 336 | 168 | 58 | 84 | 32 |
| N1 | 326 | 163 | 0 | 82 | 0 |
| N2 | 143 | 72 | 0 | 36 | 0 |
| N3 | 450 | 225 | 0 | 113 | 0 |
| N4 | 625 | 313 | 0 | 156 | 0 |
| N5 | 228 | 114 | 0 | 57 | 0 |

TABLE 7

| | | Quantity of RNA added to reaction solution of reverse transcription reaction | | | | | | | |
|---|---|---|---|---|---|---|---|---|---|
| | | 1 μg | | 0.5 μg | | 0.25 μg | | 0.125 μg | |
| | | | COX-2 | | COX-2 | | COX-2 | | COX-2 |
| | | Volume | expression | Volume | expression | Volume | expression | Volume | expression |
| | RNA | of | level | of | level | of | level | of | level |
| | concentration | addition | (number | addition | (number | addition | (number | addition | (number |
| Sample No. | (ng/μL) | (μL) | of copies) | (μL) | of copies) | (μL) | of copies) | (μL) | of copies) |
| C1 | 210 | 4.8 | 0 | 2.4 | 0 | 1.2 | 0 | 0.6 | 221 |
| C2 | 562 | 1.8 | 1 | 0.9 | 12 | 0.4 | 731 | 0.2 | 309 |
| C3 | 285 | 3.5 | 0 | 1.8 | 0 | 0.9 | 0 | 0.4 | 15 |
| C4 | 135 | 7.4 | 0 | 3.7 | 0 | 1.9 | 0 | 0.9 | 2 |
| C5 | 336 | 3.0 | 325 | 1.5 | 179 | 0.7 | 88 | 0.4 | 39 |
| N1 | 326 | 3.1 | 0 | 1.5 | 0 | 0.8 | 0 | 0.4 | 0 |
| N2 | 143 | 7.0 | 1 | 3.5 | 0 | 1.7 | 0 | 0.9 | 0 |
| N3 | 450 | 2.2 | 0 | 1.1 | 0 | 0.6 | 0 | 0.3 | 2 |
| N4 | 625 | 1.6 | 0 | 0.8 | 0 | 0.4 | 0 | 0.2 | 0 |
| N5 | 228 | 4.4 | 2 | 2.2 | 0 | 1.1 | 0 | 0.5 | 0 |

### Example 6

### Sensitivity and Specificity of MYBL2 Detection Using Clinical Specimens

The examinees consisted of 29 colon cancer patients and 29 healthy subjects. Their feces were respectively collected and then dispensed at 0.5 cc into 15 ml tubes. Thereafter, RNA was recovered from each fecal sample in the form of 50 μL of an RNA solution in the same manner as that of Example 5. The quantity of the recovered total RNA was determined by measuring the concentration of each RNA solution using the NanoDrop instrument (a product of NanoDrop Technologies, Inc).

The cDNA was synthesized from the RNA recovered in the RNA solution, by using the ReverTra Ace qPCR RT Kit (a product of Invitrogen). At this time, the synthesis was carried out under two types of reaction conditions regarding the RNA quantity for use in the reverse transcription reaction as follows: (a) a condition in which 1 μL of the RNA solution was added to the reaction solution of the reverse transcription reaction, irrespective of the concentration of the recovered RNA solution; and (b) a condition in which the quantity of the RNA solution to be added to the reaction solution was adjusted in accordance with the concentration of the RNA solution so that 1 μg of RNA could be added to the reaction solution of each reverse transcription reaction.

Thereafter, the expression level of the human MYBL2 (myeloblastosis viral oncogene homolog-like 2) was measured by PCR with use of the obtained cDNA as a template. Specifically speaking, the cDNA was added with 12.5 μL, of the 2× TaqMan PCR master mix (a product of Applied Biosystems, Inc), and a primer probe set for human MYBL2 detection (a product of Applied Biosystems, Inc) so that the final volume was set to 25 μL. By so doing, a PCR solution was prepared. The PCR solution was subjected to TaqMan PCR analysis by using the ABI Prism 7700 Sequence Detection System (a product of Applied Biosystems, Inc). The heat cycle of PCR was in accordance with the usage instruction. The quantification was conducted on the basis of the results of the fluorescence intensity obtained by using a dilution series having known concentrations of a standard plasmid as a template. It was deemed to be positive (meaning that the MYBL2 expression product was detected) if fifty or more copies were produced.

As a result, in the case of the condition (b) where the RNA quantity to be added to the reaction solution was equally set at 1 μg, the MYBL2 expression product was detected in ten cases out of 29 colon cancer patients and one case out of 29 healthy subjects (the sensitivity was 34% and the specificity was 97%). On the other hand, in the case of the condition (a) where a fixed volume of the RNA solution was added to the reaction solution irrespective of the concentration of the recovered RNA, the MYBL2 expression product was detected in fifteen cases out of 29 colon cancer patients and one case out of 29 healthy subjects, meaning better sensitivity (the sensitivity was 52% and the specificity was 97%) than the case of the condition (b). This Example showed that the analysis on the volume basis of extracted nucleic acids, like the detection method of the present invention, can achieve better test results in cases where a cancer-related gene is to be detected from cancer-cell derived nucleic acids recovered from feces together with bacteria-derived nucleic acids.

### Example 7

### Application to the Expression Analysis of Colon Cancer-Related Gene: 4

Feces of a healthy subject was mixed well to be homogenized, which was then added with MKN45 cells so that 1×10⁴ cells could be contained per gram of the fecal sample. This fecal sample was dispensed at volumes of 1 mL, 2 mL, and 3 mL, using a syringe. Four samples were prepared per each volume. The thus prepared twelve samples were weighed. The weights of four samples prepared by dispensing 1 mL were respectively 0.8 g, 0.7 g, 0.8 g, and 0.8 g, the weights of four samples prepared by dispensing 2 mL were respectively 1.7 g, 1.6 g, 1.7 g, and 1.6 g, and the weights of four samples prepared by dispensing 3 mL were respectively 2.6 g, 2.5 g, 2.6 g, and 2.5 g.

In order to recover RNA, two samples out of four samples of each volume were respectively added with an extraction solution (acid phenol guanidine solution) so that the volume would be 6 mL per gram of feces, and then suspended. The remaining two samples of each volume were respectively added with 6 mL volume of the extraction solution (acid phenol guanidine solution) per each sample irrespective of the weight of feces, and then suspended. Each of these suspensions was added with 6 mL of chloroform, and then centrifuged at 12,000×g at 4° C. for 20 minutes. A fixed quantity (2 mL) was dispensed respectively from the aqueous layer yielded from the centrifugal separation, irrespective of the quantity of the added extraction solution, and RNA was recovered therefrom in the form of 50 μL of an RNA solution by using the RNeasy midi kit (a product of Qiagen). The quantity of the recovered total RNA was determined by measuring the concentration of each RNA solution using the NanoDrop instrument (a product of NanoDrop Technologies, Inc).

The cDNA was synthesized from the RNA recovered in the RNA solution, by using a commercially available reverse transcription reaction kit (a product of Invitrogen), through a reverse transcription reaction where 1 μL of the RNA solution was added to the reaction solution of the reverse transcription reaction, irrespective of the concentration of the recovered RNA solution. The real-time PCR was carried out with use of the obtained cDNA as a template in the same protocol as that of Example 1.

As a result, the expression level (number of copies) of the COX-2 gene per 1 μL of RNA recovered from the sample was such that: out of the six samples prepared by adjusting the liquid quantity of the extraction solution as per the weight of the collected feces, two samples prepared by dispensing 1 mL respectively produced 833 copies and 786 copies, two samples prepared by dispensing 2 mL respectively produced 780 copies and 791 copies, and two samples prepared by dispensing 3 mL respectively produced 770 copies and 811 copies. On the other hand, out of the six samples prepared by adding a fixed quantity of the extraction solution irrespective of the weight of feces, two samples prepared by dispensing 1 mL respectively produced 821 copies and 816 copies, two samples prepared by dispensing 2 mL respectively produced 1582 copies and 1640 copies, and two samples prepared by dispensing 3 mL respectively produced 2445 copies and 2451 copies.

The results of the six samples prepared by adding a fixed quantity of the extraction solution irrespective of the weight of feces were corrected by the weight of feces. Specifically speaking, a correction factor was determined so that 0.8 g, the average weight of four 1 ml fecal samples, could serve as 1, and each expression level (number of copies) was divided by the correction factor. As a result, two samples prepared by dispensing 1 mL respectively showed 821(821/1) copies and 816 (816/1) copies, two samples prepared by dispensing 2 mL respectively showed 744 (1582/2.125) copies and 820 (1640/2) copies, and two samples prepared by dispensing 3 mL respectively showed 752 (2445/3.25) copies and 784 (2451/3.125) copies.

Table 8 shows the thus measured expression levels (number of copies) of the COX-2 gene. In the Table, the column (a) shows the results of the six samples prepared by adjusting the liquid quantity of the extraction solution as per the weight of the collected feces, while the column (b) shows the results of the six samples prepared by adding a fixed quantity of the extraction solution irrespective of the weight of feces.

TABLE 8

| | | Volume of feces | | | | | |
|---|---|---|---|---|---|---|---|
| | | 1 mL | | 2 mL | | 3 mL | |
| (a) | Weight of | 0.8 | 0.7 | 1.7 | 1.6 | 2.6 | 2.5 |
| | feces (g) | | | | | | |
| | COX-2 | 833 | 786 | 780 | 791 | 770 | 811 |
| | expression | | | | | | |
| | level | | | | | | |
| | (number | | | | | | |
| | of | | | | | | |
| | copies) | | | | | | |
| (b) | Weight of | 0.8 | 0.8 | 1.7 | 1.6 | 2.6 | 2.5 |
| | feces (g) | | | | | | |
| | Correction | 1 | 1 | 2.125 | 2 | 3.25 | 3.125 |
| | factor | | | | | | |
| | COX-2 | 821 | 816 | 1582 | 1640 | 2445 | 2451 |
| | expression | | | | | | |
| | level | | | | | | |
| | (number | | | | | | |
| | of | | | | | | |
| | copies) | | | | | | |
| | Corrected | 821 | 816 | 744 | 820 | 752 | 784 |
| | COX-2 | | | | | | |
| | expression | | | | | | |
| | level | | | | | | |
| | (number | | | | | | |
| | of | | | | | | |
| | copies) | | | | | | |

From these results, it was found that the difference between samples in cases of the detection of a target nucleic acid was reduced by adjusting the quantity of the extraction solution to be added to feces in proportion to the quantity of the feces. Meanwhile, it is considered to be possible to obtain more accurate values by correcting the variance in the weight of feces with respect to the obtained expression level of the target nucleic acid, as long as the initial weights of feces (feces collected for the recovery of nucleic acids) are approximately the same.

### INDUSTRIAL APPLICABILITY

The detection method of the present invention is capable of accurately, easily, and simply detecting a target nucleic acid in feces, and thus is particularly applicable to the fields of clinical tests and the like.

### BRIEF DESCRIPTION OF THE REFERENCE SYMBOLS

11: Container main body
12: Cap
13: Sampling rod
13a: Hole
13b: Movable lid
E: Feces
S: Storage solution

1. A method for detecting a target nucleic acid sequence derived from an animal which excreted feces, from a fecal sample thereof, comprising:
(a) collecting a fixed quantity of feces;
(b) recovering nucleic acids from the feces that has been collected in (a), and preparing a fixed volume of a nucleic acid solution; and
(c) dispensing a fixed volume of an aliquot from the nucleic acid solution that has been prepared in (b), and detecting the target nucleic acid sequence in the dispensed solution.

2. The method for detecting a target nucleic acid sequence derived from an animal according to claim 1, wherein said (a) is (a′) below, and the quantity of the target nucleic acid sequence detected in said (c) is corrected on the basis of the quantity of the feces collected in said (a′):
(a′) collecting feces and measuring the quantity of the feces.

3. The method for detecting a target nucleic acid sequence derived from an animal according to claim 2, wherein the correction of the quantity of the target nucleic acid sequence is conducted by dividing the quantity of the target nucleic acid sequence detected in said (c) by the quantity of the feces collected in said (a′).

4. The method for detecting a target nucleic acid sequence derived from an animal according to claim 1, wherein said (a) is (a′) below, and said (b) is (b′-1) below:
(a′) collecting feces and measuring the quantity of the feces; and
(b′-1) recovering nucleic acids from the feces that has been collected in said (a′) and preparing a nucleic acid solution of a volume proportional to the quantity of the feces collected in said (a′).

5. The method for detecting a target nucleic acid sequence derived from an animal according to claim 4, wherein said (b′-1) is (b′-2) below:
(b′-2) mixing the feces that has been collected in said (a′) or the solid content of the feces, with an extraction solution having a volume proportional to the quantity of the feces collected in said (a′), recovering a nucleic acid extracted in the extraction solution, and preparing a nucleic acid solution of a volume proportional to the quantity of the feces collected in said (a′).

6. The method for detecting a target nucleic acid sequence derived from an animal according to claim 1, wherein said (a) is (a′) below, and said (b) is (b″) below:
(a′) collecting feces and measuring the quantity of the feces:
(b″) mixing the feces that has been collected in said (a′) or the solid content of the feces, with an extraction solution having a volume proportional to the quantity of the feces collected in said (a′), extracting a nucleic acid therein, then dispensing a fixed volume of an aliquot from the extraction solution, recovering nucleic acids in the dispensed solution, and preparing a fixed volume of a nucleic acid solution.

7. The method for detecting a target nucleic acid sequence derived from an animal according to claim 2, wherein the quantity of feces is determined by at least one measurement values selected from the group consisting of a weight, a volume, a volume of the solid content of the feces, and an absorbance.

8. The method for detecting a target nucleic acid sequence derived from an animal according to claim 1, wherein said target nucleic acid is RNA.

9. The method for detecting a target nucleic acid sequence derived from an animal according to claim 1, wherein said target nucleic acid sequence is a nucleic acid sequence derived from a human.

10. The method for detecting a target nucleic acid sequence derived from an animal according to claim 1, wherein said target nucleic acid sequence is a nucleic acid sequence derived from a marker gene of a digestive organ disease.

11. The method for detecting a target nucleic acid sequence derived from an animal according to claim 1, wherein said target nucleic acid sequence is a nucleic acid sequence derived from a marker gene of cancer.

12. The method for detecting a target nucleic acid sequence derived from an animal according to claim 1, wherein said target nucleic acid sequence is a nucleic acid sequence derived from a marker gene of colon cancer.

13. A method of testing for the presence or absence of affection by a disease comprising:
determining whether or not the animal is affected by the disease from the quantity of the target nucleic acid sequence detected on the basis of a preset threshold, the quantity of the target nucleic acid sequence having been determined by collecting a fixed quantity of feces from an animal, recovering nucleic acids from the feces, and preparing a fixed volume of a nucleic acid solution, and dispensing a fixed volume of an aliquot from the nucleic acid solution, and detecting a target nucleic acid sequence in the dispensed solution,
wherein the target nucleic acid sequence is derived from a marker gene of the disease.

14. A method of testing for the presence or absence of affection by colon cancer comprising:
determining the patient who provided the feces is affected by colon cancer if the quantity of the target nucleic acid detected by the method for detecting a target nucleic acid derived from an animal according to claim 1, is equal to or greater than a preset threshold, and the patient is not affected by colon cancer if the quantity is smaller than said threshold, the quantity of the target nucleic acid having been determined by collecting a fixed quantity of feces from an animal, recovering nucleic acids from the feces, and preparing a fixed volume of a nucleic acid solution, and dispensing a fixed volume of an aliquot from the nucleic acid solution, and detecting a target nucleic acid sequence in the dispensed solution,
wherein the target nucleic acid sequence is derived from a marker gene of Colon cancer.

15. The method of testing for colon cancer according to claim 14, wherein said target nucleic acid is a nucleic acid derived from COX-2 (cyclooxygenase-2) gene.
